(19) United States
(12) Patent Application Publication
(10) Pub. No.: US 2011/0081399 A1
(43) Pub. Date: Apr. 7, 2011

(54) SURFACE MODIFICATION OF NITINOL

(75) Inventors: John Gerard O'Donoghue, Waterford (IE); Peter O'Hare, Atrim (IE)

(73) Assignee: ENBIO LIMITED, Carrigtwohill, Co. Cork (IE)

(21) Appl. No.: 12/921,950

(22) PCT Filed: Mar. 13, 2009
(86) PCT No.: PCT/GB09/00702

§ 371 Date: Dec. 6, 2010

Related U.S. Application Data
(60) Provisional application No. 61/036,109, filed on Mar. 13, 2008.

(51) Int. Cl. A61F 2/82; A61K 33/42; A61P 9/00; A61P 43/00; B05D 3/10; B05D 5/00

(52) U.S. Cl. 424/423; 424/602; 427/2.25

(57) ABSTRACT

Disclosed herein are methods of modifying a nitinol surface by using abrasive blasting techniques. The surface modification can be performed by abrasively blasting the surface and delivering at least one dopant from one or more fluid jets to cause the at least one dopant to impregnate and/or coat the nitinol surface. The nitinol surface can form a portion or all of a medical device, such as an implantable medical device, e.g., a stent.

### RELATED APPLICATIONS

This application claims the benefit of priority under 35 U.S.C. §119(e) to U.S. Prov. App. 61/036,109, filed Mar. 13, 2008, the disclosure of which is incorporated herein by reference.

### FIELD OF THE INVENTION

The present invention relates to the modification of surfaces comprising nitinol, such as nitinol stents.

### BACKGROUND OF THE INVENTION

The recent interest in surface modification technology as it relates to biomedical devices is fueled by the success of the Drug Eluting Stent (DES). Since the introduction of endovascular techniques in the 1990's revascularisation strategies have changed dramatically over the last number of years. However, in-stent restenosis (ISR) remains a problem wherein rupture of the vessel lining at the stent site can cause platelet activation, the secretion of inflammation mediators and eventually smooth muscle cell (SMC) formation, a process analogous to scar formation around a wound site. Furthermore as the stent also contacts the blood, it runs the risk of inducing a foreign body reaction in the tissue or blood cells.

DES system uses surface modification technology to combat these problems where the surface of the stent is used to deliver active agents (anti-restenosis and anti-thrombosis agents) usually in a polymer matrix locally to the device site where they are most needed. This technology was pioneered by Cordis with the Cypher® stent which received FDA approval in 2003. Since then a number of other DES have appeared on the market all aimed at reducing ISR and thrombosis in patients that have percutaneous coronary intervention (PCI) procedures. All of these active devices use a polymer matrix to carry the drug on the surface of the stent and control its elution characteristics in vivo.

However problems have arisen with the DES attributed to a number of factors, among them, achieving proper control of the elution characteristics of the drug(s). The polymer matrix (which degrades with time to release the drug and the polymer degradation products) has been identified as a possible culprit in patients with hypersensitivity. Thus, there are continuing efforts to develop new methods to control the delivery and elution of the drugs.

A large body of prior art in the stent arena has been directed towards achieving passive coatings on the stent surface to mediate ISR. These include such processes as nitriding and carbon-nitriding, the use of carbon and silicon carbide coatings as well as processes to thicken or augment the native oxide layer on the surface of the stent materials including oxidation, ion implantation and electrochemical treatments such as electropolishing or electroplating with inert metals. All such processes however have a number of disadvantages and no one treatment technique as such provides the ideal surface for optimal clinical results. For example, oxidation and ion implantation are aggressive treatments that could damage any actives (i.e., drugs or biologics) present in the coating. Coating a stent with inert metals has not produced positive outcomes in clinical trials and some such treatments have actually been found to increase restenosis. Furthermore, the polymer based drug eluting stents have also raised concerns due to the potential for long term adverse effects arising from the use of polymer carrier materials.

Accordingly, a need remains to develop new methods for modifying surfaces for stents.

### BRIEF DESCRIPTION OF THE DRAWINGS

FIG. 1 schematically depicts a method and apparatus for compressing the diameter of a stent (Example 2);

FIG. 2 shows XPS survey scans of sample POH001 from three randomly selected points (0.125 inch/sec. stent, Example 3);

FIG. 3 shows XPS survey scans of sample POH002 from three randomly selected points (0.25 inch/sec. stent, Example 4);

FIG. 4 shows XPS survey scans of sample POH003 from three randomly selected points (0.25 inch/sec. stent, Example 5);

FIGS. 5A-5D are SEM micrographs of sample POH001 (pre-stress, 0.125 inch/sec. stent) at 34× magnification (FIG. 5A), 159× magnification (FIG. 5B), 473× magnification (FIG. 5C), and 1146× magnification (FIG. 5D);

FIGS. 6A-6F are SEM micrographs of sample POH003 (post-stress, 0.25 inch/sec. stent) at 96× magnification (FIG. 6A), 113× magnification (FIG. 6B), 227× magnification (FIG. 6C), 212× magnification (FIG. 6D), 710× magnification (FIG. 6E), and 1322× magnification (FIG. 6F);

FIG. 7 is an SEM micrograph of Stent 1 before fatigue testing (Example 6);

FIG. 8 is an SEM micrograph of Stent 1 before fatigue testing (Example 6);

FIG. 9 is an SEM micrograph of Stent 3 after fatigue testing (Example 6); and

FIG. 10 is an SEM micrograph of Stent 4 after fatigue testing (Example 6).

### DETAILED DESCRIPTION

Disclosed herein are systems and treatment processes for modifying a surface of a nitinol stent with a dopant. The strength of the bond or adherence between the dopant and the nitinol surface and the concentration of dopant achieved in or on the surface can be improved over conventional methods of surface modification techniques.

The bombardment or blasting of metal surfaces with abrasive materials is finding an increasing number of technical applications in recent years. Techniques such as grit blasting, shot blasting, sand blasting, shot peening and micro abrasion fall under this category of surface treatment technique. In each of these techniques, generally, an abrasive material, shot or grit, is mixed with a fluid and delivered at high velocity to impinge the surface to be treated. The technique used to deliver the abrasive material (“abrasive blasting”) can be classified as wet or dry depending on the choice of fluid medium used to deliver the abrasive to the surface, usually water and air respectively. Abrasive blasting is typically used for cleaning or stripping substrates of an outer surface. Disclosed herein are abrasive blasting techniques to not only clean the outer surface of a stent, but to impregnate and/or coat and/or otherwise modify the stent surface with a dopant.

If a dopant material is delivered to the surface within a high velocity fluid jet in the absence of an abrasive, no or minimal surface modification will occur. Such circumstances can arise for a number of reasons; the material may not have sufficient particle size, or have sufficient density and hardness. It may also be a consequence of the nature of the surface itself. For metal surfaces, for example, the dopant material would require suitable physical properties (e.g., density and/or hardness) to breech an oxide layer to access the underlying metal surface.

In most metallic materials an oxide layer forms at the surface, which will be harder than the bulk metal or alloy. Metal surfaces (especially those of titanium and titanium derived alloy such as nitinol) are naturally contaminated in air by a variety of contaminants. The detailed physical and chemical properties of any metal surface depend on the conditions under which they are formed. The inherent reactivity of the metal can also attract various environmental chemicals/contaminants that oxidize on the surface. For example, titanium and nitinol are highly reactive metals and are readily oxidized by several different media. This results in the metal surface being covered in an oxide layer. This oxide layer is chemically stable but not always chemically inert, as the oxide layer can continue to react with various reactants in its environment, e.g., organic molecules. Modification of the metal surface/oxide layer can result in the presence of new materials in the oxide layer. In some cases the new material in the oxide layer can be advantageous to the eventual functionality of the surfaces affected; however, in some cases the new material can constitute an unwanted intrusion. (“Titanium in Medicine,” D. M. Brunette; P. Tengvall; M. Textor; P. Thompson, Springer, New York; ISBN 3-540-66936-1.)

One embodiment provides a method of modifying a nitinol stent, comprising:

abrasively blasting the surface of the nitinol stent with a plurality of abrasive particles; and

delivering at least one dopant from one or more fluids jet to impregnate and/or coat the stent with the at least one dopant.

Another embodiment provides a method of modifying a stent, comprising:

abrasively blasting the surface of the stent having a metal oxide surface with a plurality of abrasive particles to breech the metal oxide surface and expose the nitinol surface; and

delivering at least one dopant from one or more fluids jet to impregnate and/or coat the stent with the at least one dopant.

A stent is commonly a tubular structure disposed inside the lumen of a duct to relieve an obstruction. Commonly, stents are inserted into the lumen in a non-expanded form; after insertion, the stents are then expanded autonomously, or with the aid of a second device in situ. A typical method of expansion occurs through the use of a catheter-mounted angioplasty balloon that is inflated within the stenosed vessel or body passageway in order to shear and disrupt the obstructions associated with the wall components of the vessel and to obtain an enlarged lumen.

In one embodiment, the stent is used for treating narrowing or obstruction of a body passageway in a human or animal in need thereof. “Body passageway” as used herein refers to any of number of passageways, tubes, pipes, tracts, canals, sinuses or conduits which have an inner lumen and allow the flow of materials within the body. Representative examples of body passageways include arteries and veins, lacrimal ducts, the trachea, bronchi, bronchiole, nasal passages (including the sinuses) and other airways, eustachian tubes, the external auditory canal, oral cavities, the esophagus, the stomach, the duodenum, the small intestine, the large intestine, biliary tracts, the ureter, the bladder, the urethra, the fallopian tubes, uterus, vagina and other passageways of the female reproductive tract, the vasdeferens and other passageways of the male reproductive tract, and the ventricular system (cerebrospinal fluid) of the brain and the spinal cord. Exemplary devices of the invention are for these above-mentioned body passageways, such as stents, e.g., vascular stents. There is a multiplicity of different vascular stents known in the art that may be utilized following percutaneous transluminal coronary angioplasty.

In one embodiment, the abrasive blasting method results in a stent in which dopant maintains its adherence to the stent even during and after deformation, e.g., the initial crimping where the stent can be reduced in diameter, e.g., up to half of its original diameter. The crimping process is performed when the stent is mounted on a catheter, e.g., a balloon catheter. Another deformation process involves expansion of the stent after implantation in a body lumen. The stent can be expanded to approximately 2 or 3 times its diameter from a crimped state.

In one embodiment, the steps of abrasively blasting and delivering are performed substantially simultaneously. In another embodiment, the delivering can occur immediately after the abrasively blasting, e.g., before the oxide layer reforms.

In one embodiment, the abrasively blasting removes up to 10% of the nitinol stent prior to impregnating and/or coating the stent with the dopant.

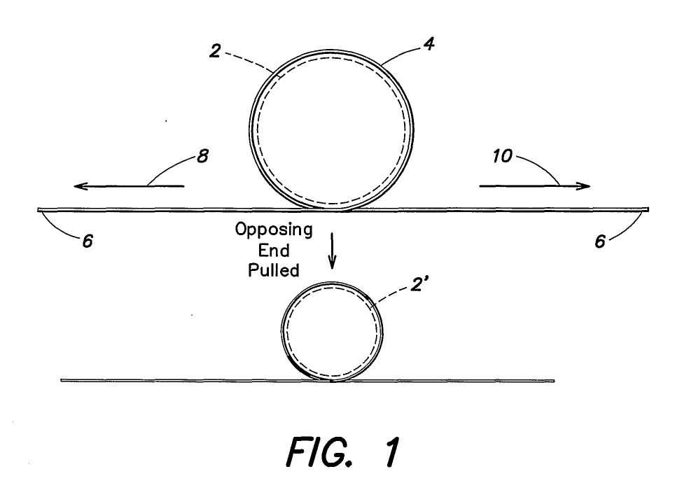

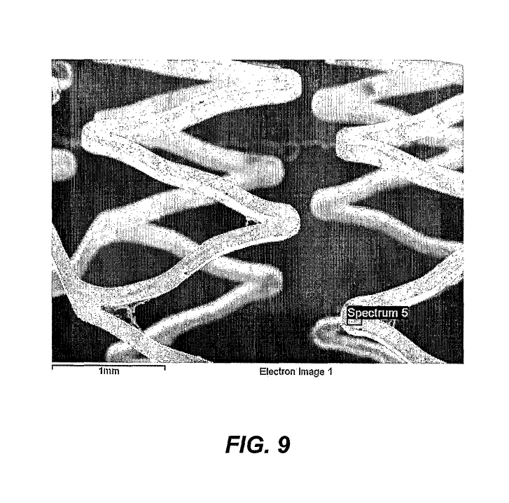

The plurality of abrasive particles and at least one dopant can be delivered from the same fluid jet nozzle or from two or more different fluid jet nozzles, where the two or more different fluid jet nozzles are positioned at the same incident angle, at different incident angles, or coaxially. Various configurations of fluid jet nozzles and methods for delivering abrasives and dopants to coat/impregnate a substrate surface with the dopants are described in PCT Publication No. WO 2008/033867, the disclosure of which is incorporated herein by reference, including the disclosures of FIGS. 1 and 9. Other fluid jet nozzles and methods for delivering abrasives and dopants are disclosed in the PCT Application entitled “NOZZLE CONFIGURATIONS FOR ABRASIVE BLASTING,” filed Mar. 11, 2009, assigned to EnBIO.

In one embodiment, the stent can be annealed prior to or after the abrasive blasting, depending on the stability of the dopant under annealing conditions.

Another embodiment provides a method of modifying a nitinol surface, comprising:

abrasively blasting the surface of the nitinol surface with a plurality of abrasive particles; and

delivering at least one dopant from one or more fluids jet to impregnate and/or coat the nitinol surface with the at least one dopant.

In one embodiment, the nitinol surface forms a portion or all of a medical device, such as an implantable medical device, e.g., a stent.

In certain embodiments, the dopant materials include but are not limited to materials desired at an implant surface for the purposes of steering and improving the body tissue-implant interaction. The dopant can comprise materials such as polymers, metals, ceramics (e.g., metal oxides, metal nitrides), and combinations thereof, e.g., blends of two or more thereof.

Exemplary dopants include, calcium phosphates and modified calcium phosphates, including Ca₅(PO₄)₃OH (hydroxyapatite), CaHPO₄.2H₂O, CaHPO₄, Ca₈H₂(PO₄)₆.5H₂O, α-Ca₃(PO₄)₂, β-Ca₃(PO₄)₂ or any calcium phosphate containing carbonate, chloride, fluoride, silicate or aluminate anions, protons, potassium, sodium, magnesium, barium or strontium cations.

Other exemplary dopants include titania (TiO₂), zirconia, hydroxyapatite, silica, carbon, and chitosan/chitin. In one embodiment, the dopant is a combination of an agent-carrying media and at least one therapeutic agent (including biomolecules and biologics). Potential carriers for therapeutic agents including antibiotics, immuno suppressants, antigenic peptides, bactericidal peptides, structural and functional proteins have been disclosed in U.S. Pat. No. 6,702,850). Calcium phosphate coatings as the drug carrier can also be used (see U.S. Pat. Nos. 6,426,114, 6,730,324, and U.S. Provisional Application No. 60/410,307, the disclosures of which are incorporated herein by reference). Dopants that can act as agent-carrying media include nanoporous, mesoporpous, nanotubes, micro-particles of various materials including hydroxyapatite, silica, carbon, and titania (TiO₂) capable of carrying therapeutic agents, biomolecules and biologics. Particulates and powders (e.g. titania powder) can be either adhesively bonded or covalently attached (tethered) to the therapeutic agents, biomolecules and biologics. In an another embodiment, the therapeutic agents, biomolecules or biologics can be physically encapsulated within the carrier in the form of microspheres.

Composites of media and carriers (e.g. sintered together), and combinations of carriers can convey drugs and biologics and can control elution profiles.

Other exemplary dopants include barium titanate, zeolites (aluminosilicates), including siliceous zeolite and zeolites containing at least one component selected from phosphorous, silica, alumina, zirconia, calcium carbonate, biocompatible glass, calcium phosphate glass. The dopant can also be a growth factor consisting of epidermal growth factors, transforming growth factor α, transforming growth factor β, vaccinia growth factors, fibroblast growth factors, insulin-like growth factors, platelet derived growth factors, cartilage derived growth factors, interlukin-2, nerve cell growth factors, hemopoietic cell growth factors, lymphocyte growth factors, bone morphogenic proteins, osteogenic factors or chondrogenic factors.

In one embodiment, the dopant is a calcium phosphate or modified calcium phosphate (e.g., hydroxyapatite) deposited on the surface of the nitinol stent. In another embodiment, both HA and a metal oxide coat the surface of the stent. Both HA and the metal oxide constitute excellent biocompatible biointerfaces, both being biostable and safe in the body. Both can be termed bioreactive in that they can induce specific responses in certain tissues particularly bone tissue. The surface resulting from the deposition of HA on titanium as delivered by the micro-blasting technique combines the benefits of both materials. In the event that the nitinol stent is not fully covered by the dopant (HA), the oxide-coated portions of the stent presents a biocompatible surface to the biological tissue, while the HA affixed on and in the surface is not denatured by the deposition process and therefore conveys its full benefit to the surrounding tissue. In this manner the different benefits of both biomaterials can brought to bear in the biointerface and when further combined with the surface texture/morphology best suited to intended functionality of the implant, and moreover the availability of a drug delivery mechanism, can provide various methods for tailoring the therapeutic, compositional and morphological profile available to the patient end user.

In one embodiment, the dopant is a therapeutic agent. The therapeutic agent can be delivered as a particle itself, or immobilized on a carrier material. Exemplary carrier materials include any of the other dopants listed herein (those dopants that are not a therapeutic agent) such as polymers, calcium phosphates and modified calcium phosphates (as disclosed herein), titanium dioxide, silica, biopolymers, biocompatible glasses, zeolite, demineralized bone, de-proteinated bone, allograft bone, and composite combinations thereof.

Exemplary classes of therapeutic agents include anti-cancer drugs, anti-inflammatory drugs, immunosuppressants, an antibiotic, heparin, a functional protein, a regulatory protein, structural proteins, oligo-peptides, antigenic peptides, nucleic acids, immunogens, and combinations thereof.

In one embodiment, the therapeutic agent is chosen from antithrombotics, anticoagulants, antiplatelet agents, thrombolytics, antiproliferatives, anti-inflammatories, antimitotic, antimicrobial, agents that inhibit restenosis, smooth muscle cell inhibitors, antibiotics, fibrinolytic, immunosuppressive, and anti-antigenic agents.

In one embodiment, agents that inhibit restenosis include sirolimus, paclitaxel, tacrolimus, heparin, pimecrolimus, and everolimus.

In one embodiment, the dopant is a radio opaque material, such as those chosen from alkalis earth metals, transition metals, rare earth metals, and oxides, sulphates, phosphates, polymers and combinations thereof.

In one embodiment, the carrier material is a biopolymer selected from polysaccharides, gelatin, collagen, alginate, hyaluronic acid, alginic acid, carrageenan, chondroitin, pectin, chitosan, and derivatives, blends and copolymers thereof.

In one embodiment, the dopant is delivered in a gaseous carrier fluid, such as nitrogen, hydrogen, argon, helium, air, ethylene oxide, and combinations thereof. In another embodiment, the dopant is delivered in a liquid carrier fluid. In one embodiment, the liquid is also an etching liquid (basic or acidic). In one embodiment, the dopant is delivered in an inert environment.

Another embodiment relates to the chemical treatment of metal surfaces for the purposes of adhesion. Good adhesion of paints and polymeric coatings to metal surfaces is an area of increasing technical importance. This technology can be used to pre-treat a surface by impregnating it with compounds having desired chemical functionality. These include but are not limited to polymers or silica materials having siloxane groups.

The pretreatment can be used to lay down a very strongly bound layer of seed polymer material on the surface. Further polymer coatings could then be attached to this seed layer rather than trying to attaching it directly to the surface of the metal.

The dopant is not limited to one compound but could be any combination of any of the materials listed or even any material(s) that do(es) not have the necessary mechanical properties to impregnate the surface if delivered singularly at high velocity to the surface.

In one embodiment, the dopant can be any material so long as it is passive, i.e., unreactive with the surface. It simply has to be at the surface when the oxide layer is breached by the abrasive so that the oxide reforms around it.

In one embodiment, the dopant is nanocrystalline.

In one embodiment, the dopant is nanocrystalline hydroxyapatite.

In one embodiment the abrasive has a suitable property chosen from at least one of size, shape, hardness, and density to break the oxide layer. In one embodiment, the abrasive has a modus hardness (Mohs hardness) ranging from 0.1 to 10, such as a modus hardness ranging from 1 to 10, or a modus hardness (Mohs hardness) ranging from 5 to 10. In another embodiment, the abrasive has a particle size ranging from 0.1 μm to 10000 μm, such as a particle size ranging from 1 μm to 5000 μm, or a particle size ranging from 10 μm to 1000 μm.

Abrasive materials to be used in this invention include but are not limited to shot or grit made from silica, alumina, zirconia, barium titanate, calcium titanate, sodium titanate, titanium oxide, glass, biocompatible glass, diamond, silicon carbide, calcium phosphate and modified calcium phosphate, calcium carbonate, metallic powders, carbon fiber composites, polymeric composites, titanium, stainless steel, hardened steel, carbon steel, chromium alloys, apatite grit (e.g., MCD grit, Himed, NY), and combinations thereof.

The pressure of the fluid jet will also be a factor in determining the impact energy of the abrasive. The abrasive and dopant(s) do not have to be delivered to the surface through the same jet. They could be in any number of separate jets as long as they deliver the solid components to the surface at the substantially the same time, e.g., prior to reformation of the oxide layer if the surface is a metal. This allows a large amount of flexibility in optimizing the invention towards a specific need. In one embodiment, the fluid jet is selected from wet blasters, abrasive water jet peening machines, and wet shot peening machines. In one embodiment, the at least one fluid jet operates at a pressure ranging from 0.5 to 100 bar, such as a pressure ranging from 1 to 30 bar, or a pressure ranging from 1 to 10 bar.

In another embodiment, the at least one fluid jet is selected from dry shot peening machines, dry blasters, wheel abraders, grit blasters), sand blasters(s), and micro-blasters. In one embodiment, the at least one fluid jet operates at a pressure ranging from 0.5 to 100 bar, such as a pressure ranging from 1 to 30 bar, or a pressure ranging from 3 to 10 bar.

In other embodiments, blasting equipment can be used in conjunction with controlled motion such as CNC or robotic control. The blasting can be performed in an inert environment.

In one embodiment the abrasive material is alumina (10 Mesh) while the dopant is HA with a particle size range of 0.1 to 3 μm. The mixed media is achieved by mixing the dopant and abrasive between the ratio of 5:95 and 95:5 HA to Silica volume % but more preferably between the ratio of 80:20 to 20:80 and most preferably in the ratio range 60:40 to 40:60. The silica bead has a Mohs hardness in the range of 0.1 to 10 but most preferably in the range of 2 to 10 and most preferably in the range 5 to 10. This mixed media is delivered to a titanium surface using a standard grit blasting machine operating in the pressure range of 0.5 Bar to 20 Bar, such as a pressure range of 2 to 10 bar, or a pressure range of 4 Bar to 6 Bar. The distance between the nozzle and the surface can be in the range of 0.1 mm to 100 mm, such as a range of 0.1 mm to 50 mm, or a range of 0.1 mm to 20 mm. The angle of the nozzle to the surface can range from 10 degrees to 90 degrees, such as a range of 30 degrees to 90 degrees, or a range of 70 to 90 degrees.

In another embodiment the abrasive material is silica (10 Mesh) while the dopant is HA with a particle size range of 0.1 to 3 μm. The mixed media is achieved by mixing the dopant and abrasive between the ratio of 5:95 and 95:5 HA to alumina weight % but more preferably between the ratio of 80:20 to 20:80 and most preferably in the ratio range 60:40 to 40:60. The Alumina grit has a Mohs hardness in the range of 0.1 to 10, such as a range of 2 to 10, or a range of 5 to 10. This mixed media can be delivered to a titanium surface using a standard grit blasting machine operating in the pressure range 0.5 Bar to 20 Bar, such as a pressure range of 2 to 10 bar, a range of 4 Bar to 6 Bar. The distance between the nozzle and the surface can range from 0.1 mm to 100 mm, such as a range of 0.1 mm to 50 mm, or a range of 0.1 mm to 20 mm. The angle of the nozzle to the surface can range from 10 degrees to 90 degrees, such as a range of 30 degrees to 90 degrees, or a range of 70 to 90 degrees.

One of ordinary skill in the art can appreciate the influence of machine parameters including jet velocity, operating pressure, venturi configuration, angle of incidence and surface to nozzle distances on the extent of impregnation of the dopant in the surface using these mixed media.

One of ordinary skill in the art can appreciate the effect of the size, shape, density and hardness of the abrasive material used on the extent of impregnation of the dopant in the surface using these mixed media.

One of ordinary skill in the art can appreciate the effect of the fluid stream itself, the blasting equipment using a gas medium (typically air) the effects of using inert gases as a carrier fluid e.g. N2 or noble gases such as Ar and He on the extent of impregnation of the dopant in the surface using these mixed media.

In the case of wet blasting equipment using a liquid as a carrier fluid (normally water), One of ordinary skill in the art can appreciate the effect of acidity and basicity on the extent of impregnation of the dopant in the surface using these mixed media.

As disclosed herein, the disclosed methods can be useful for modifying the surfaces of nitinol stents. In the context of stent applications, dopants can be active (eliciting a biological response) or passive (not eliciting a biological response). Passive dopants can be conveyed to enhance lubricity or render a substrate radio-opaque, of enhance wear characteristics or enhance adhesion of an ad-layer, etc. Active agents can evoke a response from the host tissue in vivo, enhancing the functionality of the device or the surgery, or delivering a benefit as a secondary function to the device.

One embodiment provides a method of treating at least one disease or condition associated with vascular injury or angioplasty comprising, implanting in a subject in need thereof a nitinol stent impregnated with a calcium phosphate or a modified calcium phosphate.

In one embodiment, the at least one disease or condition is a proliferative disorder, e.g., restenosis, a tumor, or the proliferation of smooth muscle cells. In one embodiment, the at least one disease or condition is an inflammatory disease. In one embodiment, the at least one disease or condition is an autoimmune disease.

### EXAMPLES

### Example 1

This example describes the surface modification of two Nitinol stents by delivering Hydroxyapatite as the dopant in one particle stream and alumina bead as the abrasive in a separate particle stream using a twin micro-blaster setup.

An experiment was conducted by loading the blasting materials into the reservoirs of two separate Comco Accuflo Micro-blasters. Each micro-blaster feeds a separate nozzle housed in a Comco Advanced Lathe. The two nozzles are mounted on an adjustable blast head configured to synchronize the flow from each nozzle to the same point on the stent surface. The stents were mounted on a rotating spindle, while the blasting head moved over the length of the stent.

100 micron particle size Alumina bead-Alox, (Mohs hardness 9, Comco Inc.) was used in all test runs. The synthetic Hydroxyapatite (lot #: 260-07205, Manufacturer: SAI, France) used had a particle size range of 25 to 60 microns. The laser cut Nitinol stents were purchased from Comco Inc. (Comco use same for R&D and process development purposes)

The parameters were set as follows:

HA blast pressure: 60 PSI;

Alox blast pressure: 60 PSI;

HA nozzle ID: 0.030 inch;

Alox nozzle ID: 0.030 inch;

HA nozzle angle to surface: 80 degrees

Alox nozzle angle to surface: 80 degrees

Nozzle distance from surface: 0.5 inch

Spindle speed: 120 rpm

Blast head feed rate: 0.125 inches/sec for the 1^{st} Stent

Blast head feed rate: 0.250 inches/sec for the 2^{nd} Stent

Each stent was subjected to one pass only.

The stents were then subjected to a cleaning treatment involving 20 minutes ultrasonic washing in deionized water to remove any material that was not intimately affixed to the surface. After the ultrasonic cleaning the stents were rinsed with deionized water and air-dried in an oven at 40° C. for one hour.

Samples were submitted for SEM (Scanning Electron Microscopy) and XPS. The second stent was submitted for further SEM and XPS analysis following 1000 mechanical compression cycles of the analysis to determine that delamination of the deposited HA surface modification did not occur.

### Example 2

This example describes the testing and characterization of the nitinol stents of Example 1.

The NiTistents of Example 1 were subjected to mechanical stresses by compression of the stent down to approximately 4 mm in stent diameter from the pre-stressing diameter of 8 mm, as illustrated in FIG. 1. This deformation was achieved by placing the stent 2 in an acetate “sling” device 4, which comprises an acetate looping around the stent 2. After stent 2 was placed in the loop of the acetate sling 4, the opposing ends 6 were pulled in the direction of arrows 8 and 10 to cause the stent to contract, resulting in compressed stent 2′. In this Example, the stent was compressed to approximately 4 mm in diameter and then expanded to return to its natural unstressed state of 8 mm in diameter. The compression/decompression steps represent one cycle, which was repeated in this Example 1000 times.

The set of samples analyzed by XPS is provided in Table 1 below. The description of each sample relates to the information received.

TABLE 1
HA blasted stents analysed by XPS

| Sample No: | SAMPLE DESCRIPTION |
|---|---|
| POH001 | 0.125 inch/sec. stent. |
| POH002 | 0.25 inch/sec. stent |
| POH003 | 0.250 inch/sec stent post stress |

The XPS and SEM analysis methods were chosen to identify the chemical, micro structural and surface characteristics of the hydroxyapatite (HA) coated stents.

### XPS Analysis:

X-Ray Photoelectron Spectroscopy (XPS) was carried out using a Kratos Axis Ultra DLD spectrometer. The operating conditions employed were consistent with those used previously in similar analytical studies. Spectra were recorded by employing monochromated Al Kα X-rays (hν=1486.6 electron volts (eV) operating at 15 kV and 10 mA in an ultrahigh vacuum (UHV) condition of approximately 1×10⁻⁸ Torr. A hybrid lens mode was employed during analysis (electrostatic and magnetic) to give an analysis area of approximately 300 μm×700 μm at a photoemission take off angle of 90° with respect to the sample surface. Wide energy survey scans (WESS) were obtained over the 0-1300 eV binding energy (BE) range at a pass energy of 160 eV. High resolution spectra were recorded for C1s (278-295 eV), O1s (525-540 eV), Ca2p (340-362 eV), P2p (125-140 eV) at a pass energy of 20 eV. The integral charge neutralisation system was employed during the analysis of the samples with the filament current set at 1.8 A and the charge balance to 3.6 V. Any uncorrected sample charging effects on the measured BE positions (eV) were further corrected by the conventional technique of setting the lowest BE component of the C1s spectral envelope to 285.0 eV, i.e. the value generally accepted for adventitious carbon surface contamination.

### XPS Results:

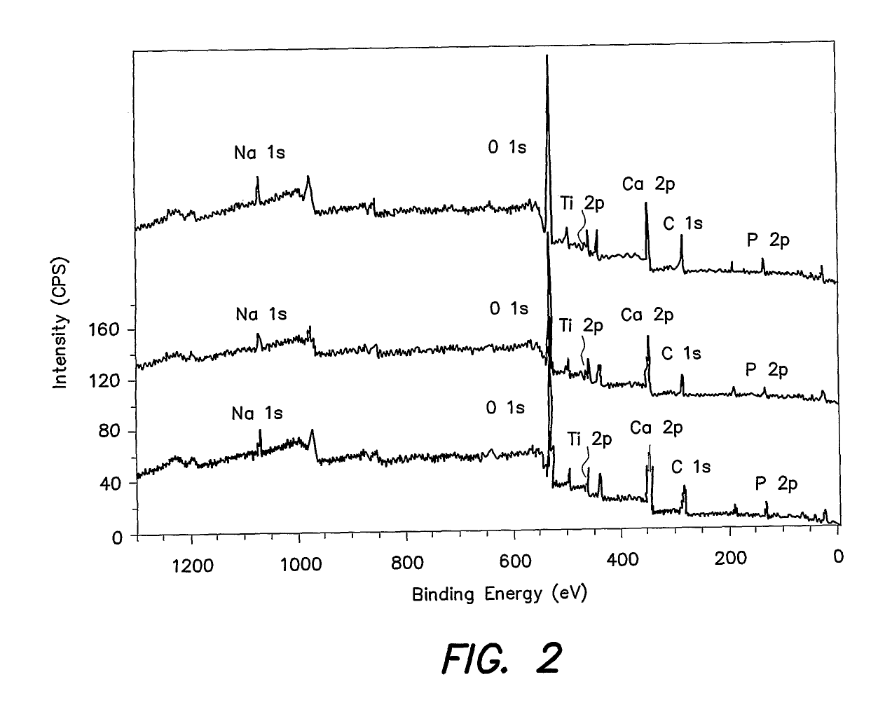

The XPS wide energy survey scans and associated high resolution plots recorded at three randomly selected points for each of the samples are shown in FIGS. 2-4. To carry out quantitative analysis on the XPS data, the photoelectron spectra were further processed by subtracting a linear background and using the peak area for the most intense spectral line of each of the detected elemental species to obtain the relative % atomic concentrations as recorded in Tables 2-4. Data, including the Ca/P ratio is reported from the results of an analysis of three individual areas on each sample surface.

### Example 3

### POH001—0.125 Inch/Sec. Stent (Pre Stress)

FIG. 2 shows three XPS survey scans of sample POH001 (0.125 inch/sec. stent), the data of which is shown in Table 2.

TABLE 2
Data derived from the quantification of elements contained
within the 0.125 inch/sec. stent.

| | | Atomic Concentration | | |
|---|---|---|---|---|
| POH001 | | (%) | | |
| | Position BE | Point | Point | Point |
| Peak | (eV) | A | B | C |
| Na 1s | 1071 | 2.65 | 2.92 | 3.47 |
| O 1s | 532.5 | 51.72 | 52.18 | 52.24 |
| Ti 2p | 455.5 | 2.66 | 2.89 | 2.30 |
| Ca 2p | 346.5 | 10.75 | 10.30 | 10.83 |
| C 1s | 285 | 27.35 | 27.76 | 26.69 |
| P 2p | 133.5 | 4.87 | 3.95 | 4.46 |

| | | | |
|---|---|---|---|
| CaP ratio | 2.21 | 2.61 | 2.43 |

The resultant peaks obtained from this sample show XPS peaks associated with calcium (Ca2p) and phosphorus (P2p). The resulting Ca/P ratios from the three points of analysis ranged from 2.21 to 2.61 (cf. HA at 1.67). The main contributions to the spectrum are oxygen (O1s) and carbon (C1s). A small amount of titanium was also detected 2.3 to 2.89 at %). A weak but detectable signal attributable to sodium is also detected.

### Example 4

### POH002—0.25 Inch/Sec. Stent (Pre Stress)

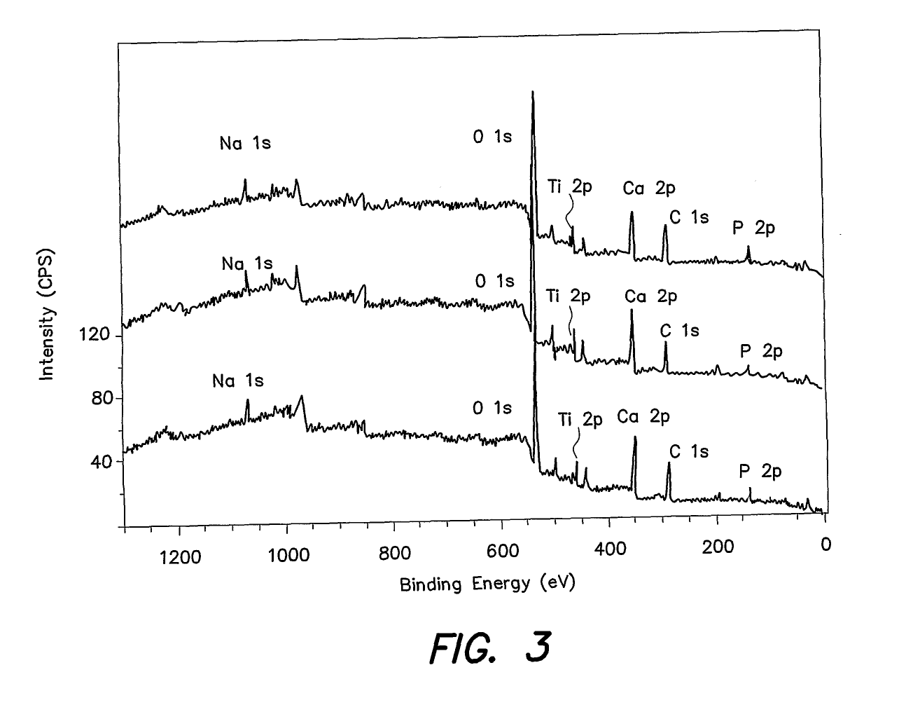

FIG. 3 shows three XPS survey scans of sample POH002 (0.25 inch/sec. stent), the data of which is shown in Table 3.

TABLE 3
Data derived from the quantification of elements contained
within the 0.25 inch/sec. stent.

| | | Atomic Concentration | | |
|---|---|---|---|---|
| POH002 | | (%) | | |
| | Position BE | Point | Point | Point |
| Peak | (eV) | A | B | C |
| Na 1s | 1071 | 2.82 | 2.65 | 1.76 |
| O 1s | 532.5 | 47.15 | 48.76 | 48.49 |
| Ti 2p | 455.5 | 2.59 | 3.15 | 2.97 |
| Ca 2p | 346.5 | 8.51 | 8.56 | 7.27 |
| C 1s | 285 | 32.88 | 33.24 | 36.28 |
| P 2p | 133.5 | 6.06 | 3.65 | 3.23 |

| | | | |
|---|---|---|---|
| CaP ratio | 1.40 | 2.35 | 2.25 |

The resultant peaks obtained from this sample show XPS peaks associated with calcium (Ca2p) and phosphorus (P2p). The resulting Ca/P ratios from the three points of analysis ranged from 1.40 to 2.35 (cf. HA at 1.67). The main contributions to the spectrum are oxygen (O1s) and carbon (C1s). A small amount of titanium was also detected 2.59 to 3.15 at %). A weak but detectable signal attributable to sodium is also detected.

### Example 5

### POH003—0.25 Inch/Sec. Stent (Post Stress)

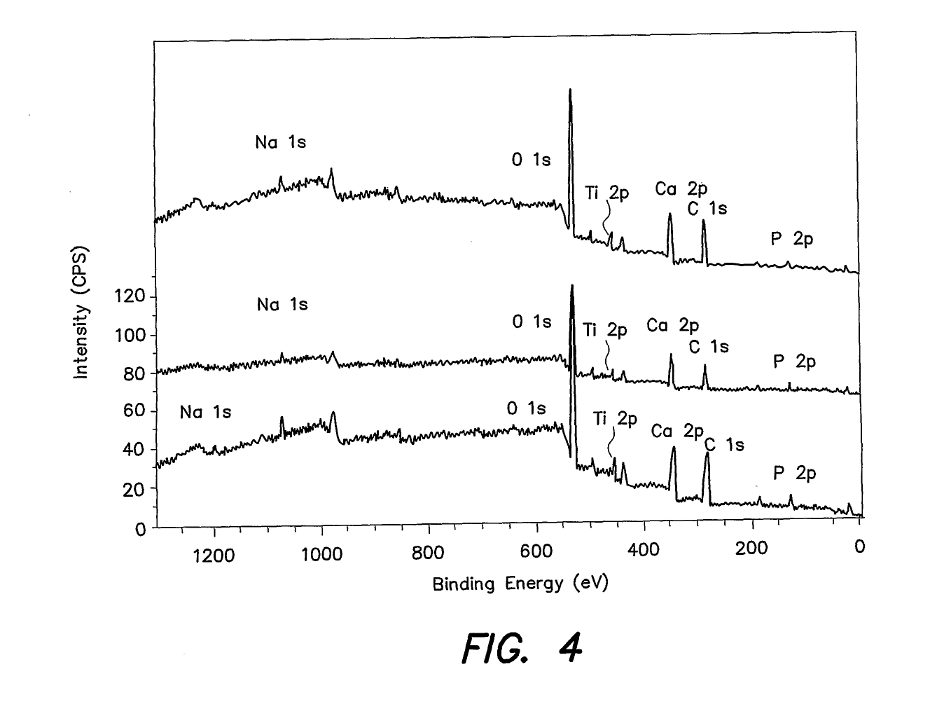

FIG. 4 shows three post stress XPS survey scans of sample POH003 (0.25 inch/sec. stent), the data of which is shown in Table 4.

TABLE 4
Data derived from the quantification of elements contained
within the 0.25 inch/sec. stent post stress.

| Post stress stent | | Atomic Concentration | | |
|---|---|---|---|---|
| (0.25 inch/sec.) | | (%) | | |
| | Position BE | Point | Point | Point |
| Peak | (eV) | D | E | F |
| Na 1s | 1071 | 1.76 | 2.10 | 1.27 |
| O 1s | 532.5 | 48.49 | 45.79 | 42.50 |
| Ti 2p | 455.5 | 2.97 | 1.84 | 1.71 |
| Ca 2p | 346.5 | 7.27 | 9.34 | 9.36 |
| C 1s | 285 | 36.28 | 36.90 | 42.32 |
| P 2p | 133.5 | 3.23 | 4.03 | 2.83 |

| | | | |
|---|---|---|---|
| CaP ratio | 2.25 | 2.32 | 3.31 |

POH003 Post Stress Analysis (0.25 Inch/Sec. Stent)

The resultant peaks obtained from this sample show XPS peaks associated with calcium (Ca2p) and phosphorus (P2p). The resulting Ca/P ratios from the three randomly selected points of analysis ranged from 2.25 to 3.31 (cf. HA at 1.67). The main contributions to the spectrum are oxygen (O1s) and carbon (C1s). A small amount of titanium was also detected 1.71 to 2.97 at %). A weak but detectable signal attributable to sodium is also detected.

Analysis of the XPS spectra and corresponding quantitative data reported here for surface of HA blasted stents indicates the presence of the elements expected: calcium phosphate materials with a Ca/P ratio greater than for stoichiometric HA. There is some variation in the Ca/P ratio calculated from data attained from different areas on the same sample; notably, however, two of the three randomly selected points for the pre-stress and post-stress stents show similar values, i.e., points B and C of the pre-stress stent and points D and E of the post-stress stent. In many of the samples, signals from metal elements (Titanium) attributable to the substrate surface are also detected.

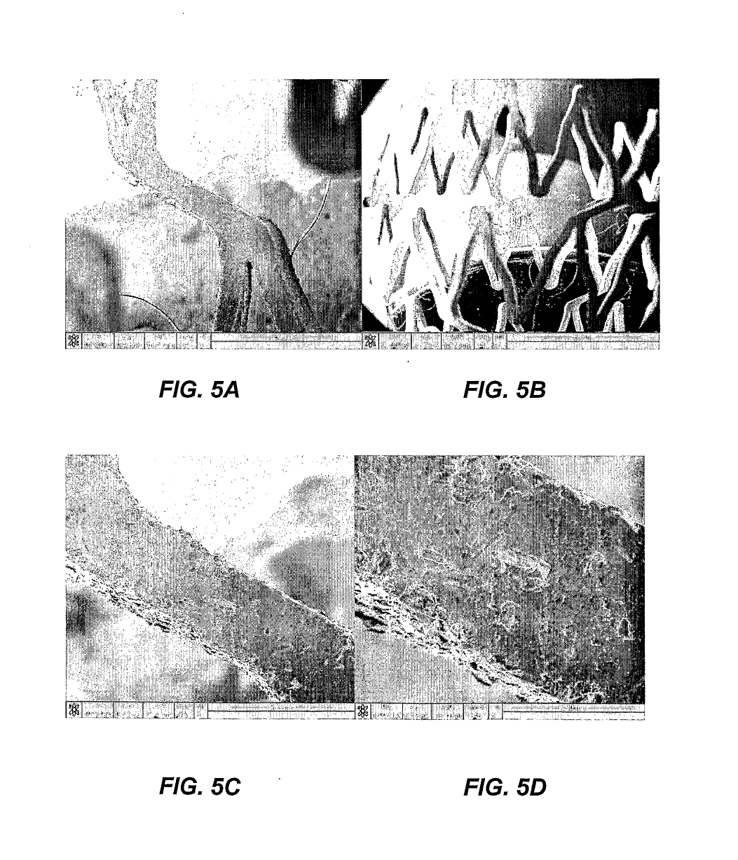

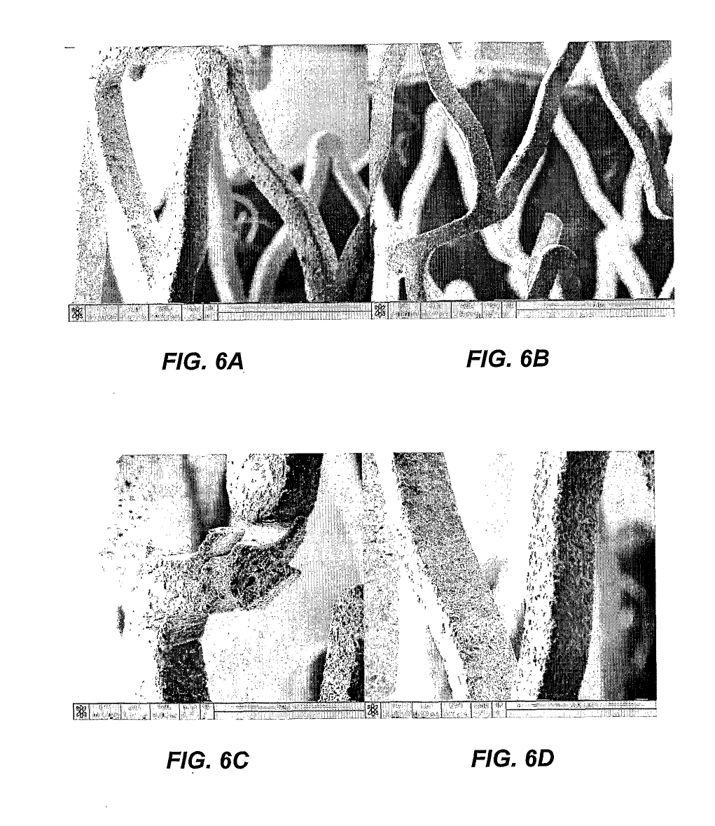

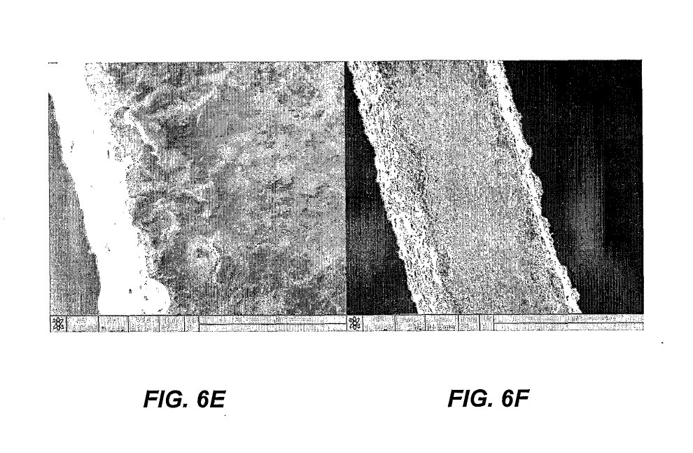

FIGS. 5 and 6 show SEM micrographs of sample POH001 (0.125 inch/sec. stent) and sample POH003 (0.25 inch/sec. stent post stress).

### Example 6

Nitinol stents were sourced from Lumenous Device Technologies (Sunnyvale, Calif.). The stents were constructed from superelastic straight annealed Nitinol (A_{f} Temp: approx 15-20 Deg C.) of the following dimensions:

OD: 8 mm

Length: 20 mm.

Hydroxyapatite (HA) was deposited onto the surface of the nitinol stents using a Comco standard micro-blast lathe (twin nozzle). The height of the blast nozzle was 20 mm from the surface and was moved over the substrate at a speed of 1 mm/second as the stent rotated at a speed of 30 mm/sec. The abrasive powder (MCD grit from Himed, NY) was flowed at a pressure of 60 psi and the HA (supplied by SAI, France) was flowed at 90 psi to produce a treated surface. Stent 1 was analysed by SEM-EDX to determine surface coating coverage and Stents 2-4 subjected to fatigue testing according to the industry standard test EN 14299:2004: Non-active surgical implants. Particular requirements for cardiac and vascular implants. Specific requirements for arterial stents.

This benchtop fatigue test was intended to provide empirical evidence of the structural integrity of coated devices when subjected to mechanical fatigue replicating in vivo conditions. The test was designed to simulate the device radial fatigue due to expansion and contraction of the vessel surrounding it. Physiological strain of a healthy vessel was modelled using silicone arteries implanted with the device. The test was accelerated to obtain results in a shorter time period than physiological rates would allow, frequency of 60 Hz. Test cycle parameters, resulting in 0.1-5% physiological compliance, were determined in a physiologically simulating silicone tube (ID: 7-8 mm). Each cycle applied pulsatile stresses within the tubes to simulate the circumferential strain at the in vivo application site. Test duration imitated 1-2 years of implantation life at 72 bpm. Testing was conducted under simulated physiological conditions: saline at 37° C.+/−2°. This test demonstrated the integrity of the HA coated stent under mechanical fatigue failures for a minimum of 1-2 years post-implantation. A device failure was defined as any broken or cracked geometry (struts, crowns, links, etc.) visible at 30× magnification during or at the end of the test and through SEM.

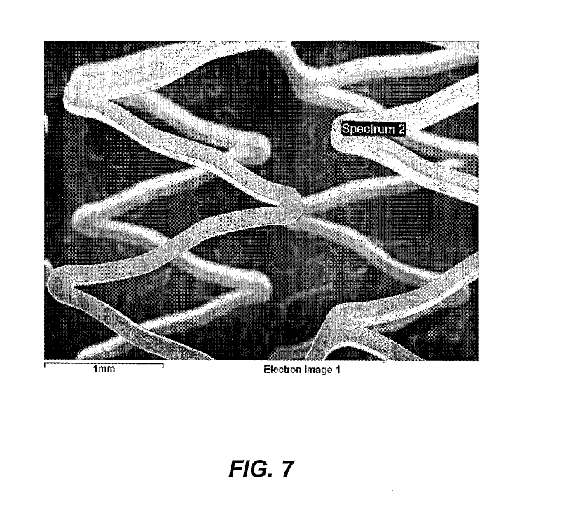

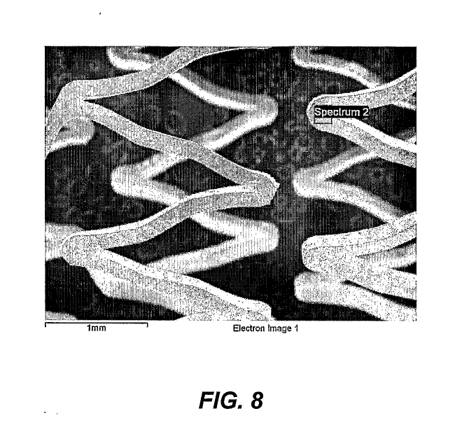

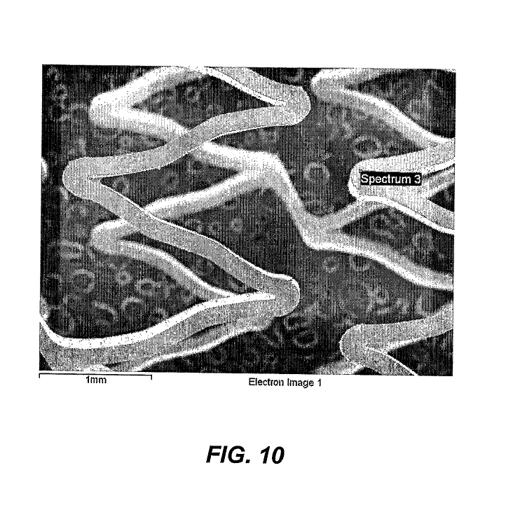

The microscopic examination of the fatigue tested stents revealed no signs of damage to the stent, even under SEM magnification. Furthermore, there was no sign of coating delamination or coating damage as a result of the fatigue testing, as shown in FIGS. 7-10.

The chemical composition of a single sample (Stent 1) was analysed in the as-coated condition (not subjected to fatigue test) and this was compared to the chemical composition of the fatigue tested samples (Stents 2-4) using SEM/EDX. This visual and elemental analysis was conducted to evaluate the coating level & integrity. Analysis of the devices before and after testing are shown in Table 5. (Average taken from 6 points on outer surface area of device). The elemental composition of the fatigue tested samples is largely similar to the composition of the as coated stent, indicating that the coating has not been removed by the fatigue testing and is still intimately attached to the nitinol surface. Of particular note is the composition of Ni and Ti in the analysis, as an increase in the level of these elements in the analysis would suggest that the coating has been removed. In all cases, the level of each element is in the 4-6% level which suggests that the level of coating coverage has not been changed by the fatigue testing.

### CONCLUSIONS

Following examination of the devices post fatigue testing no fatigue failures (cracks or fractures of stent struts) were found. The addition of the HA Coating had no structural affect on the device after 50 million cycles.

TABLE 5
SEM-EDX analysis of Stent 1 (without fatigue testing) and of the
fatigue tested stents (Stents 2-4)

| Element | Stent 1 | Stent 2 | Stent 3 | Stent 4 |
|---|---|---|---|---|
| C (Carbon) | 9.21 | 13.81 | 17.99 | 13.68 |
| O (Oxygen) | 63.33 | 60.43 | 59.60 | 61.78 |
| P (Phosphorous) | 7.52 | 6.66 | 6.35 | 6.38 |
| Ca (Calcium) | 9.01 | 7.47 | 7.50 | 7.08 |
| Ti (Titanium) | 5.86 | 6.24 | 4.58 | 5.82 |
| Ni (Nickel) | 5.07 | 5.39 | 3.98 | 5.27 |

1. A method of modifying a nitinol stent, comprising:
abrasively blasting the surface of the nitinol stent with a plurality of abrasive particles; and
delivering at least one dopant from one or more fluids jet to impregnate and/or coat the stent with the at least one dopant.

2. A method as claimed in claim 1, wherein the at least one dopant is a therapeutic agent.

3. A method as claimed in claim 1, wherein the abrasively blasting comprises delivering the plurality of abrasive particles from one or more fluid jets, wherein the fluid jet can be the same or different from the at least one fluid jet that delivers the at least one dopant.

4. A method as claimed in claim 1, wherein the abrasively blasting removes up to 10% of the nitinol stent prior to impregnating and/or coating the stent with the dopant.

5. A method as claimed in claim 1, wherein the abrasively blasting and the delivering occur substantially simultaneously.

6. A method as claimed in claim 1, wherein the stent is annealed prior to the step of abrasively blasting.

7. A method as claimed in claim 1, wherein the stent is annealed after the step of abrasively blasting.

8. A method as claimed in claim 1, wherein the at least one dopant is a polymeric material.

9. A method as claimed in claim 1, wherein the step of abrasively blasting is selected from grit blasting, micro blasting, water jet blasting, and shot peening.

10. A method as claimed in claim 1, wherein the step of abrasively blasting is performed by delivering the abrasive particles from one or more fluid jets.

11. (canceled)

12. A method as claimed in claim 10, wherein the one or more fluid jets are selected from dry shot peening machines, dry blasters, wheel abraders, grit blasters, sand blasters(s), and micro-blasters.

13-24. (canceled)

25. A method as claimed in claim 1, wherein the abrasively blasting is performed with an abrasive selected from silica, alumina, zirconia, barium titanate, calcium titanate, sodium titanate, titanium oxide, glass, biocompatible glass, diamond, silicon carbide, calcium phosphate, modified calcium phosphate, calcium carbonate, metallic powders, metallic wires, carbon fiber composites, polymers, polymeric composites, titanium, stainless steel, hardened steel, chromium alloys, apatite grit, and combinations thereof.

26. A method as claimed in claim 1, wherein the abrasively blasting is performed with an abrasive having a Mohs hardness ranging from 0.1 to 10.

27-31. (canceled)

32. A method as claimed in claim 2, wherein the at least one therapeutic agent is an osteoconductive or osteointegrative agent.

33. A method as claimed in claim 32, wherein the osteoconductive or osteointegrative agent is selected from calcium phosphates and modified calcium phosphates.

34. A method as claimed in claim 32, wherein the osteoconductive or osteointegrative agent is selected from Ca₅(PO₄)₃OH, CaHPO₄.2H₂O, CaHPO₄, Ca₈H₂(PO₄)₆.5H₂O, α-Ca₃(PO₄)₂, β-Ca₃(PO₄)₂, and combinations thereof.

35. A method as claimed in claim 33, wherein the modified calcium phosphate contains at least one anion selected from carbonate, chloride, fluoride, silicate and aluminate.

36. A method as claimed in claim 33, wherein the modified calcium phosphate contains at least one cation selected from protons, potassium, sodium, magnesium, barium and strontium.

37. A method as claimed in claim 2, wherein the therapeutic agent is delivered as immobilized on or in a carrier material.

38. A method as claimed in claim 37, wherein the carrier material is selected from polymers, calcium phosphate, modified calcium phosphate, titanium dioxide, silica, biopolymers, biocompatible glasses, zeolite, demineralised bone, de-proteinated bone, allograft bone, and composite combinations thereof.

39-54. (canceled)

55. A nitinol stent having a surface modified according to the method of claim 1.

56. A nitinol stent having a calcium phosphate and/or modified calcium phosphate coating at least a portion of a surface thereof.

57. A nitinol stent as claimed in claim 56, wherein the calcium phosphate and/or modified calcium phosphate is hydroxyapatite.

58. A nitinol stent impregnated and/or coated with a calcium phosphate and/or modified calcium phosphate.

59. A nitinol stent as claimed in claim 58, wherein the calcium phosphate and/or modified calcium phosphate is hydroxyapatite.

60. A nitinol stent as claimed in claim 56, wherein the calcium phosphate and/or modified calcium phosphate further comprises a therapeutic agent.

61. A method of treating at least one disease or condition associated with vascular injury or angioplasty comprising, implanting in a subject in need thereof a nitinol stent impregnated and/or coated with a calcium phosphate and/or modified calcium phosphate.
